(19) United States
(12) Patent Application Publication
(10) Pub. No.: US 2009/0111868 A1
(43) Pub. Date: Apr. 30, 2009

(54) Use Of Rottlerin And Its Derivatives As Activators Of BK Channel For Therapy Of Hypertension And Related Disorders

(76) Inventors: Steven O. Marx, New York, NY (US); Sergey I. Zakharov, Fort Lee, NJ (US)

Correspondence Address: Kevin C. Hooper;Bryan Cave LLP, 1290 Avenue of the Americas, New York, NY, 10104, US

(21) Appl. No.: 11/792,202

(22) PCT Filed: Nov. 18, 2005
(86) PCT No.: PCT/US05/41942

§ 371 Date: Mar. 4, 2008

Related U.S. Application Data
(60) Provisional application No. 60/632,487, filed on Dec. 2, 2004.

(51) Int. Cl. A61K 31/352; C07D 311/04

(52) U.S. Cl. 514/456; 549/405

(57) ABSTRACT

The present invention provides compositions and methods for regulating the BK channel using rottlerin and derivatives thereof. In particular, the present invention provides pharmaceutical compositions for use in treating or preventing BK channel medicated disorders including hypertension and various hyperexcitability disorders. Also provided are compositions and methods for use in post-stroke neuroprotection and in treating or preventing erectile dysfunction. The present invention further provides kits for use in treating or preventing BK channel mediated disorders, comprising the compositions of the present invention.

### BACKGROUND OF THE INVENTION

The high incidence of stroke and hypertension in the United States remains a leading indication for visits to physicians, the use of prescription drugs and morbidity/mortality. It is estimated that more than 50 million Americans (approximately one third of the adult population) currently suffer from hypertension. Two-thirds of the population over age 70 suffers from hypertension. Chronic blood pressure elevation leads to end-organ damage, including the eye, cardiac, and central nervous system. Greater understanding of the molecular mechanisms leading to the regulation of membrane excitability in both brain and the vasculature may have important implications in improving therapeutic modalities.

The BKCa channel complex plays a critical role in regulating contractile tone in smooth muscle and the vasculature (Brenner, et al., Vasoregulation by the betal subunit of the calcium-activated potassium channel. Nature, 2000. 407(6806): 870-6; Brayden, et al., Regulation of arterial tone by activation of calcium-dependent potassium channels. Science, 1992. 256(5056):532-5). Furthermore, neuronal BKCa channel function is not well-studied, yet it remains clear that the channel has significant effects on neurotransmitter release and neuronal discharges (Robitaille, et al., Functional colocalization of calcium and calcium-gated potassium channels in control of transmitter release. Neuron, 1993. 11(4):645-55). Thus, the BKCa channel represents an important integrator of signal transduction pathways, potently mediating cellular excitability in a diverse group of cell types. Recent studies have suggested that the channel may have a role in innate immunity in neutrophils. (Ahluwalia, et al., The large-conductance Ca2+-activated K+channel is essential for innate immunity. Nature, 2004. 427(6977):853-8), recognition as a heme-binding protein (Tang, et al., Haem can bind to and inhibit mammalian calcium-dependent S1o1 BK channels. Nature, 2003. 425(6957):531-5), behavior responses to ethanol (Davies, et al., A central role of the BK potassium channel in behavioral responses to ethanol in C. elegans. Cell, 2003. 115(6):655-66) and function as a protective mechanism against ischemically-driven cell death in cardiac myocytes (Xu, et al., Cytoprotective role of Ca2+-activated K+channels in the cardiac inner mitochondrial membrane. Science, 2002. 298(5595):1029-33). These novel functions of the BKCa channel remain to be further validated and explored (Moczydlowski, E. G., BK Channel News: Full Coverage on the Calcium Bowl. J. Gen. Physiol., 2004: 123(5):471-3). Utilizing molecular biologic and electrophysiologic approaches, the inventors of the present invention are seeking to elucidate the mechanism(s) through which the BKCa channel is allosterically regulated. By unraveling the complex mechanism(s) mediating phosphorylation-dependent and β1 subunit regulation of the channel, the inventors seek to identify specific regions that are responsible for activation and inhibition of channel function. Novel approaches for the treatment of disorders ranging from neurologic dysfunction (seizures, memory) to vascular complications related to diabetes or hypertension will follow from elucidation of the basic mechanism(s) mediating BKCa channel activity.

### Regulation of Blood Pressure and VSMC Contractility by Ca_{y}1.2, RyR and BKCa Channels

Arterial blood pressure is determined by several factors, including vascular tone, which represents the contractile activity of smooth muscle within the walls of resistance vessels. The contractile state of smooth muscle is organized through the interplay of vasoconstrictor and vasodilatory neurohormones and by blood pressure itself (the Bayliss effect; constriction of the vessel after an increase in transmural pressure) (Bayliss, W. M., On the local reactions of the arterial wall to changes of internal pressure. J. Physiol., 1902. 28:220-23; Nelson, M. T., Bayliss, myogenic tone and volume-regulated chloride channels in arterial smooth muscle. J. Physiol., 1998. 507 (Pt 3):629; Nelson, et al., Noradrenaline contracts arteries by activating voltage-dependent calcium channels. Nature, 1988. 336(6197):382-5). The autoregulatory Bayliss effect is based upon graded membrane depolarization in response to pressure, which activates voltage dependent Ca²⁺ channels, causing vasoconstriction. Vascular smooth muscle contraction is triggered by Ca²⁺/calmodulin dependent phosphorylation of the regulatory myosin light chain. Increased intracellular Ca²⁺ is mediated by Ca²⁺ influx through Caᵥ1.2 and Ca²⁺ release from intracellular stores, mainly through the IP3R (Davis, et al., Signaling mechanisms underlying the vascular myogenic response. Physiol. Rev., 1999. 79(2):387-423). Caᵥ1.2 was recently reported to play a critical role in regulating smooth muscle contraction/blood pressure regulation, as an inducible smooth muscle specific Caᵥ1.2 knockout demonstrated abnormal autoregulation and maintenance of vascular tone in response to depolarization and pressure (Bayliss effect) (Moosmang, et al., Dominant role of smooth muscle L-type calcium channel Cav1.2 for blood pressure regulation. Embo. J., 2003. 22(22):6027-34). In vascular smooth muscle, the dynamic range of [Ca²⁺]ᵢ is narrow, ranging from˜100 nM when the artery is maximally dilated to 350 nM when arteries are maximally constricted (Knot, et al., Regulation of arterial diameter and wall [Ca2+] in cerebral arteries of rat by membrane potential and intravascular pressure. J. Physiol., 1998. 508:199-209).

Spontaneous transient outward currents (STOC) were first described in smooth muscle by Bolton and coworkers (Benham, et al., Spontaneous transient outward currents in single visceral and vascular smooth muscle cells of the rabbit. J. Physiol., 1986. 381:385-406; Bolton, et al., Spontaneous transient outward currents in smooth muscle cells. Cell Calcium, 1996. 20(2): 141-52) and have been shown in a diverse group of vascular and non-vascular smooth muscle (Hisada, et al., Properties of membrane currents in isolated smooth muscle cells from guinea-pig trachea. Pflugers Arch., 1990. 416(1-2):151-61; Ohya, et al., Cellular calcium regulates outward currents in rabbit intestinal smooth muscle cell. Am. J. Physiol., 1987. 252(4 Pt 1):C401-10; Saunders, et al., Spontaneous transient outward currents and Ca(++)-activated K+channels in swine tracheal smooth muscle cells. J. Pharmacol. Exp. Ther., 1991. 257(3): 1114-20; Nelson, et al., Relaxation of arterial smooth muscle by calcium sparks. Science, 1995. 270(5236):633-637; Nelson, et al., Physiological roles and properties of potassium channels in arterial smooth muscle. Am. J. Physiol., 1995. 268((Cell Physiol. 37)):C799-C822; Hume, et al., Macroscopic K+currents in single smooth muscle cells of the rabbit portal vein. J. Physiol., 1989. 413:49-73; Jaggar, et al., Ca2+channels, ryanodine receptors and Ca(2+)-activated K+ channels: a functional unit for regulating arterial tone. Act.a Physiol. Scand., 1998. 164(4):577-87; Porter, et al., Frequency modulation of Ca2+ sparks is involved in regulation of arterial diameter by cyclic nucleotides. Am. J. Physiol., 1998. 274(5 Pt 1):C1346-55). Each transient outward current represents the activation of 10-100 BKCa channels (Porter, et al., Frequency modulation of Ca2+ sparks is involved in regulation of arterial diameter by cyclic nucleotides. Am. J Physiol., 1998. 274(5 Pt 1):C1346-55). Nelson and colleagues obtained the first evidence of Ca²⁺ sparks in smooth muscle (Nelson, et al., Relaxation of arterial smooth muscle by calcium sparks. Science, 1995. 270(5236):633-637) and similar findings have been shown in numerous smooth muscle cells derived from arteries, portal vein, urinary bladder, gastro-intestinal tract, airway and gallbladder (Mironneau, et al., Ca2+ sparks and Ca2+ waves activate different Ca(2+)-dependent ion channels in single myocytes from rat portal vein. Cell Calcium, 1996. 20(2): 153-60; Gordienko, et al., Crosstalk between ryanodine receptors and IP(3) receptors as a factor shaping spontaneous Ca(2+)-release events in rabbit portal vein myocytes. J. Physiol., 2002. 542(Pt 3):743-62; Herrera, et al., Voltage dependence of the coupling of Ca(2+) sparks to BK(Ca) channels in urinary bladder smooth muscle. Am. J. Physiol. Cell Physiol., 2001. 280(3):C481-90; Ji, et al., Stretch-induced calcium release in smooth muscle. J. Gen. Physiol., 2002. 119(6):533-44; Ji, et al., RYR2 proteins contribute to the formation of Ca(2+) sparks in smooth muscle. J. Gen.Physiol., 2004. 123(4):377-86; Gordienko, et al., Variability in spontaneous subcellular calcium release in guinea-pig ileum smooth muscle cells. J. Physiol., 1998. 507 (Pt 3):707-20; Kirber, et al., Relationship of Ca2+ sparks to STOCs studied with 2D and 3D imaging in feline oesophageal smooth muscle cells. J. Physiol., 2001. 531 (Pt 2):315-27). Ca²⁺ sparks are transient local increases in intracellular Ca²⁺ that occur through the coordinated opening of a group of RyR located on the SR (Nelson, et al., Relaxation of arterial smooth muscle by calcium sparks. Science, 1995. 270(5236):633-637). In cerebral artery myocytes, Ca²⁺ sparks lead to activation of the BKCa channel, thus providing an important feedback role in the regulation of pressure-induced constriction (Nelson, et al., Relaxation, of arterial smooth muscle by calcium sparks. Science, 1995. 270(5236):633-637). Vasodilators may act in part through increasing the frequency of Ca²⁺ sparks. All three RyR isoforms have been reported in smooth muscle (Marks, et al., Molecular cloning and characterization of the ryanodine receptor/junctional channel complex cDNA from skeletal muscle sarcoplasmic reticulum. Proc. Natl. Acad. Sci., 1989. 86:8683-8687; Hakamata, et al., Primary Structure and distribution of a novel ryanodine receptor/calcium release channel from rabbit brain. FEBS, 1992. 312:229-235; Ledbetter, et al., Tissue distribution of ryanodine receptor isoforms and alleles determined by reverse transcription polymerase chain reaction. Journal of Biological Chemistry, 1994. 269(50):31544-51) although the relative proportion of each isoform varies between tissues (Xu, et al., Evidence for a Ca(2+)-gated ryanodine-sensitive Ca2+ release channel in visceral smooth muscle. Proc. Natl. Acad. Sci. USA, 1994. 91(8):3294-8). The physiologic role of each of the isoforms of the RyR is lacking. The respective roles of RyR2 and RyR1 in smooth muscle have been incompletely elucidated (Takeshima, et al., Excitation-contraction uncoupling and muscular degeneration in mice lacking functional skeletal muscle ryanodine-receptor gene. Nature, 1994. 369(6481):556-9; Takeshima, et al., Ca(2+)-induced Ca2+ release in myocytes from dyspedic mice lacking the type-1 ryanodine receptor. Embo. J., 1995. 14(13):2999-3006), in part because RyR2 null mice are lethal (Takeshima, et al., Embryonic lethality and abnormal cardiac myocytes in mice lacking ryanodine receptor type 2. Embo. J., 1998. 17(12):3309-16). In rat portal vein myocytes, antisense oligonucleotides targeting each of the RyR isoforms demonstrated that both RyR1 and RyR2 are required for myocytes to respond to membrane depolarization with Ca²⁺ sparks and global increase in intracellular Ca²⁺ (Coussin, et al., Requirement of ryanodine receptor subtypes 1 and 2 for Ca(2+)-induced Ca(2+) release in vascular myocytes [In Process Citation] J. Biol. Chem., 2000. 275(13):9596-603).

The RyR (Ca²⁺ spark)-BKCa channel complex can be viewed as a mechanism to limit smooth muscle contraction. Ca²⁺ spark frequency is increased when intravascular pressure is elevated from 10 to 60 mm Hg in rat cerebral arteries (Jaggar, J. H., Intravascular pressure regulates local and global Ca(2+) signaling in cerebral artery smooth muscle cells. Am. J. Physiol. Cell Physiol., 2001. 281(2):C439-48). Inhibition of RyR or BKCa channels has been demonstrated to lead to pressure-induced cerebral artery constriction (Gollasch, et al., Ontogeny of local sarcoplasmic reticulum Ca2+ signals in cerebral arteries: Ca2+ sparks as elementary physiological events [published erratum appears in Circ. Res. 1999 Jan 8-22;84(1):125]. Circ. Res., 1998. 83(11):1104-14; Knot, et al., Ryanodine receptors regulate arterial diameter and wall [Ca2+] in cerebral arteries of rat via Ca2+-dependent K+ channels. J. Physiol. (Lond), 1998. 508(Pt 1):211-21). BKCa channel from VSMC derived from β1 subunit knockout animals demonstrated˜100 fold lower probability of opening and Ca²⁺ spark induced BKCa channel current was significantly reduced and>⅓ of sparks failed to elicit a BKCa channel activation (Brenner, et al., Vasoregulation by the b1 subunit of the calcium-activated potassium channel. Nature, 2000. 407:870-876). Mean arterial pressure was elevated in the β1 subunit null animals, leading to left ventricular hypertrophy (Brenner, et al., Vasoregulation by the b1 subunit of the calcium-activated potassium channel. Nature, 2000. 407:870-876). Thus, the ability of the BKCa channel to sense the Ca²⁺ sparks was impaired by the loss of the β1 subunit. In contrast, a gain of function mutation of β1 (G352A) was associated with a low prevalence of moderate and severe diastolic hypertension (Fernandez-Fernandez, et al., Gain-of-function mutation in the KCNMB1 potassium channel subunit is associated with low prevalence of diastolic hypertension. J. Clin. Invest., 2004. 113(7):1032-9). BKCa-β1_{E65K} channels showed increased Ca²⁺ sensitivity (Fernandez-Fernandez, et al., Gain-of-function mutation in the KCNMB1 potassium channel subunit is associated with low prevalence of diastolic hypertension. J. Clin. Invest., 2004. 113(7):1032-9). Activation of the PKA and PKG signal transduction pathways leads to 2-3 fold increases in both Ca²⁺ spark and BKCa channel activity (Porter, et al., Frequency modulation of Ca²⁺ sparks is involved in regulation of arterial diameter by cyclic nucleotides. Am. J. Physiol., 19918. 274(5 Pt 1):C1346-55; Wellman, et al., Role of phospholamban in the modulation of arterial Ca(2+) sparks and Ca(2+)-activated K(+) channels by cAMP. Am. J. Physiol. Cell Physiol., 2001. 281(3):C1029-37). Ryanodine reduced dilation to forskolin by 80%, consistent with the importance of Ca²⁺ sparks and a potential regulatory role of PKA. However, in arterial smooth muscle derived from phospholamban null mice, forskolin had little effect compared to the˜2 fold increase in Ca²⁺ spark frequency in wild type animals (Wellman, et al., Role of phospholamban in the modulation of arterial Ca(2+) sparks and Ca(2+)-activated K(+) channels by cAMP. Am. J. Physiol. Cell Physiol., 2001. 281(3):C1029-37).

### Modulators of BKCa Channel Activity

Pharmacologic approaches to activate BKCa channels represent a new/emerging strategy to control membrane excitability. Despite the increasing number of natural and synthetic BKCa channel openers, relatively little is known about the interaction sites and mechanism of action. Moreover, many of the compounds are relatively weak, with non-specific activity towards BKCa channel (Ohwada, et al., Dehydroabietic acid derivatives as a novel scaffold for large-conductance calcium-activated K+ channel openers. Bioorg. Med. Chem. Lett., 2003. 13(22):3971-4). Small natural or synthetic products could have effectiveness in diseases mediated through muscular and neuronal hyperexcitability such as asthma, urinary incontinence/bladder spasm, gastroenteric hypermotility, hypertension, coronary spasm, psychoses, convulsion and anxiety (Calderone, V., Large-conductance, ca(2+)-activated k(+) channels: function, pharmacology and drugs. Curr. Med. Chem., 2002. 9(14):1385-95; Pelaia, et al., Potential role of potassium channel openers in the treatment of asthma and chronic obstructive pulmonary disease. Life Sci., 2002. 70(9):977-90; Gribkoff, et al., The pharmacology and molecular biology of large-conductance calcium-activated (BK) potassium channels. Adv. Pharmacol., 1997. 37:319-48; Nardi, et al., Natural modulators of large-conductance calcium-activated potassium channels. Planta. Med., 2003. 69(10):885-92). Recent work has suggested a role for K⁺channel activators for post-stroke neuroprotection, erectile dysfunction and cardiac diseases such as coronary artery vasospasm/hypertension (Nardi, et al., Natural modulators of large-conductance calcium-activated potassium channels. Planta. Med., 2003. 69(10):885-92; Gribkoff, et al., Targeting acute ischemic stroke with a calcium-sensitive opener of maxi-K potassium channels. Nat. Med., 2001. 7(4):471-7). The synthetic benzimidazolone derivatives NS004 and NS1619 are the pioneer BK-activators (activate BKCa current at 10-30 μM in vascular and non-vascular smooth muscle) (Coghlan, et al., Recent developments in the biology and medicinal chemistry of potassium channel modulators: update from a decade of progress. J. Med. Chem. 2001. 44(11):1627-53) and have led to the design of several novel and heterogenous BK-openers (Olesen, et al., NS 004-an activator of Ca(2+)-dependent K+ channels in cerebellar granule cells. Neuroreport, 1994. 5(8):1001-4; Olesen, et al., Selective activation of Ca(2+)-dependent K+ channels by novel benzimidazolone. Eur. J. Pharmacol., 1994. 251(1):53-9). In addition to BKCa channel opening, NS-1619 inhibits Ca²⁺ and Cl⁻ channel (Gribkoff, et al., The pharmacology and molecular biology of large-conductance calcium-activated (BK) potassium channels. Adv. Pharmacol., 1997. 37:319-48), but has been reported to increase intracellular Ca²⁺ concentration (at 30 μM in porcine coronary myocytes through the ryanodine receptor sensitive storage sites (Yamamura, et al., BK channel activation by NS-1619 is partially mediated by intracellular Ca2+release in smooth muscle cells of porcine coronary artery. Br. J. Pharmacol., 2001. 132(4):828-34). NS1608 caused BKCa channel activation (minimum effective concentration 0.5 μM; maximum between 5-10 μM), but demonstrated a bell shaped concentration with an inhibitory effect at higher concentrations (50 μM) in porcine coronary artery cells (Hu, et al., Differential effects of the BKCa channel openers NS004 and NS1608 in porcine coronary arterial cells. Eur. J. Pharmacol., 1995. 294(1):357-60; Hu, et al., On the mechanism of the differential effects of NS004 and NS1608 in smooth muscle cells from guinea pig bladder. Eur. J. Pharmacol., 1996. 318(2-3):461-8). BMS-204352 (MaxiPost) has been evaluated in clinical trials for stroke therapy and a reduction in brain infarct size has been detected in rat stroke models (Gribkoff; et al., Targeting acute ischemic stroke with a calcium-sensitive opener of maxi-K potassium channels. Nat. Med., 2001. 7(4):471-7; Imaizumi, et al., Molecular basis of pimarane compounds as novel activators of large-conductance Ca(2+)-activated K(+) channel alpha-subunit. Mol. Pharmacol., 2002. 62(4):836-46). The effects of BMS-204352 were Ca²⁺ sensitive; at 50 nM intracellular Ca²⁺, the compound had almost no effect, whereas at higher intracellular Ca²⁺ concentrations, it produced progressively greater increases in current (Gribkoff, et al., Targeting acute ischemic stroke with a calcium-sensitive opener of maxi-K potassium channels. Nat. Med., 2001. 7(4):471-7). Three glycosylated triterpenes called dehydrosoyasaponin-I (DHS-I), soyasaponins I and III have been shown to activate BKCa channels. DHS-I has poor membrane permeability, but is probably metabolized to other active molecules that penetrate the cell. DHS-I increases channel activity when the α and β subunits are co-expressed (McManus, et al., An activator of calcium-dependent potassium channels isolated from a medicinal herb. Biochemistry, 1993. 32(24):6128-33; Giangiacomo, et al., Mechanism of maxi-K channel activation by dehydrosoyasaponin-I. J. Gen. Physiol., 1998. 112(4):485-501). Maxikdiol, a 1,5-dihydroxyisoprimane diterpenoid has limited membrane permeability, but can activate the channel (threshold-1 μM; significant effect-3-10 μM) when applied to the cytoplasmic side (Nardi, et al., Natural modulators of large-conductance calcium-activated potassium channels. Planta. Med., 2003. 69(10):885-92; Singh, et al., Maxikdiol: a novel dihydroxyisoprimane as an agonist of maxi-K channels. J. Chem. Soc. Perkin Trans., 1994. 1:3349-3352; Kaczorowski, et al., High-conductance calcium-activated potassium channels; structure, pharmacology, and function. J. Bioenerg. Biomembr., 1996. 28(3):255-67; Lawson, K., Potassium channel openers as potential therapeutic weapons in ion channel disease. Kidney Int., 2000. 57(3):838-45).

### Rottlerin

Rottlerin (mallotoxin), a natural product from Mallotus phillippinensis, is a 5,7-dihydroxy-2,2-dimethyl-6-(2,4,6-trihydroxy-3-methyl-51acetylbenzyl)-8-cinnamoyl-1,2-chromene that has been frequently used as a PKCδ inhibitor based upon an in vitro study demonstrating that the IC₅₀ for PKCδ and CaMK III were 3-6 μM compared to 30-100 μM for other PKC isozymes, PKA and casein kinase II (Gschwendt, et al., Rottlerin, a novel protein kinase inhibitor. Biochem. Biophys. Res. Commun., 1994. 199(1):93-8). Based upon rottlerin, a role for PKCδ in a variety of biological events including apoptosis, cell differentiation, mitogen activated protein kinase activation and other cell processes was described. However, more recent data suggest that rottlerin is ineffective in blocking PKCδ activity in vitro, but can uncouple mitochondria (10 μM) in intact cells and reduce ATP levels in a PKC independent fashion (Soltoff, S. P., Rottlerin is a mitochondrial uncoupler that decreases cellular ATP levels and indirectly blocks protein kinase Cdelta tyrosine phosphorylation. J. Biol. Chem., 2001. 276(41):37986-92), potentially sensitizing colon carcinoma cells to tumor necrosis factor-related apoptosis (Tillman, et al., Rottlerin sensitizes colon carcinoma cells to tumor necrosis factor-related apoptosis-inducing ligand-induced apoptosis via uncoupling of the mitochondria independent of protein kinase C. Cancer Res., 2003. 63(16):5118-25). In standard assays at 0.1 mM ATP (or even 0.01 mM), rottlerin (20 μM) had virtually no effect on PKCα or PKCδ activity in the presence of phosphatidylserine (PS) using either histone H1 or myelin basic protein as a substrate (Davies, et al., Specificity and mechanism of action of some commonly used protein kinase inhibitors. Biochem. J., 2000. 351(Pt 1):95-105). Rottlerin has also been reported to inhibit insulin-induced glucose uptake (IC₅₀ 10 μM) in 3T3-L1 adipocytes (Kayali, et al., Rottlerin inhibits insulin-stimulated glucose transport in 3T3-L1 adipocytes by uncoupling mitochondrial oxidative phosphorylation. Endocrinology, 2002. 143(10):3884-96). Rottlerin has been reported to decrease the capacity for the glutamate-aspartate transporter (GLAST) subtype of the glutamate transporter (Susarla, et al., Rottlerin, an inhibitor of protein kinase Cdelta (PKCdelta), inhibits astrocytic glutamate transport activity and reduces GLAST immunoreactivity by a mechanism that appears to be PKCdelta-independent J. Neurochem., 2003. 86(3):635-45).

The history/development of rottlerin as a therapeutic compound is complex. Kamala or rottlerin has been used in India for centuries as an antihelmintic; it is violently purgative in full doses, occasionally causing nausea but seldom vomiting (Gujral, et al., Oral contraceptives. Part II. Antifertility effect of Mallotus philippinensis Mueller-argoviensis. Indian J. Med. Res., 1960. 48:52-8). In 1910, Semper reported that kamala caused a paralyzing effect on motor nerves and muscle (Gujral, et al., Oral contraceptives. Part II. Antifertility effect of Mallotus philippinensis Mueller-argoviensis. Indian J. Med. Res., 1960. 48:52-8), which based on the inventors' preliminary data is likely due to its effects on the BKCa channel. Kamala has been used in India against the tapeworm and is given to patients (3.9-11.6 grams) suspended in water, mucilage or syrup. The worm is usually expelled at the 3^{rd} or 4^{th} stool. As an external remedy, kamala is used by the people of India for various afflictions of the skins, particularly scabies. Rottlerin appears to have been isolated 150 years ago (1855) by Anderson (Anderson, A., Kamala resin-rottlerin. Edin. New Phil. Jour., 1855. 1:296-300), by allowing a concentrated ethereal solution of kamala to stand for two days, draining and pressing the granular crystals in bibulous paper and purifying them from the adhering resin. Perkin and Perkin confirmed the substance and called it mallotoxin (Gujral, et al., Oral contraceptives. Part II. Antifertility effect of Mallotus philippinensis Mueller-argoviensis. Indian J. Med. Res., 1960. 48:52-8). The pure rottlerin has the chemical composition C₃₃H₃₀O₉. Rottlerin has been given to animals; it has been shown to reduce the fertility rate of rats and guinea pigs (dose of purified rottlerin=10-20 mg/kg/day×6 days) (Gujral, et al., Oral contraceptives. Part II. Antifertility effect of Mallotus philippinensis Mueller-argoviensis. Indian J. Med. Res., 1960. 48:52-8). Injection of rottlerin (5 μM) into the cistema magna in a canine subarachnoid hemorrhage model inhibited the initial phase of cerebral vasospasm, which was attributed to its effects on PKCδ (Nishizawa, et al., Attenuation of canine cerebral vasospasm after subarachnoid hemorrhage by protein kinase C inhibitors despite augmented phosphorylation of myosin light chain. J. Vasc. Res., 2003. 40(2):169-78). No toxic effects of rottlerin were observed. It is conceivable that some of the beneficial effects may have been secondary to the effects on BKCa channel. The RTECS database (AM6913800) indicates no information regarding LD₅₀/LC₅₀ for acute/chronic toxicity.

### SUMMARY OF THE INVENTION

The inventors of the present invention disclose herein that the rottlerin and derivatives thereof are potent activators of the BK channel and that hypertension and related disorders can be treated or prevented via regulation of the BK channel using rottlerin. Accordingly, the present invention provides compositions and methods for regulating the BK channel using rottlerin and derivatives thereof.

In one aspect of the present invention, a compound for use in modulating BK channel activity is provided having the general formula:

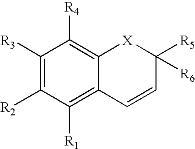

wherein X is CH2, O, N, or S; R1 and R3 is H, OH, NH or SH; R2 is ethanone, acetyl, alkenyl, aryl or alkyl; 4 is CO-[(E)CHCH]n-Ph, CN-[(E)CHCH]n-Ph, or COOZ, wherein Z is alkenyl, aryl, or alkyl; R5 and R6 is H, OH, NH, SH, alkenyl, aryl, or alkyl. In one embodiment, the compound is rottlerin or a derivative thereof.

The present invention also provides a pharmaceutical composition comprising the above-described compound, or a derivative thereof, and optionally, a pharmaceutically acceptable carrier, for use in treating or preventing a BK channel associated disorder. In a specific embodiment, the compound is rottlerin.

In another aspect of the present invention, the above-described compounds and pharmaceutical compositions can be used to regulate membrane excitability both in vitro and in vivo. In one example, the compounds and pharmaceutical compositions of the present invention can be used to treat or prevent a hyperexcitability disorder. In an embodiment of the invention, the hyperexcitability disorder is asthma. In another embodiment of the invention, the hyperexcitability disorder is hypertension. In other embodiments of the present invention, the hyperexcitability disorder includes, but is not necessarily limited to urinary incontinence, gastroenteric hypermotility, coronary spasm, psychoses, convulsion and anxiety. In another embodiment, the compounds and pharmaceutical compositions of the present invention are used in treating or preventing erectile dysfunction. In yet other embodiments, the compounds and pharmaceutical inventions of the present invention are used in treating or preventing coronary artery vasospasm and hypertension. In another embodiment, the compounds and pharmaceutical inventions of the present invention are used in treating or preventing neurologic dysfunction. In an additional embodiment, the compounds and pharmaceutical inventions of the present invention are used in post-stroke neuroprotection.

The present invention also provides methods for treating or preventing a hyperexcitability disorder in a subject, comprising administering to the subject a therapeutically effective amount of the pharmaceutical composition of the invention. In an embodiment of the invention, the hyperexcitability disorder includes, but is not necessarily limited to, asthma, urinary incontinence, gastroenteric hypermotility, hypertension, coronary spasm, psychoses, convulsion and anxiety.

The present invention also provides methods for treating or preventing erectile dysfunction in a subject by administering to the subject a therapeutically effective amount of the pharmaceutical compositions of the invention. Additionally, the present invention also provides methods for treating or preventing a coronary artery vasospasm in a subject, comprising administering to the subject a therapeutically effective amount of the pharmaceutical compositions of the present invention.

The invention additionally provides methods for treating or preventing hypertension in a subject, comprising administering to the subject a therapeutically effective amount of the pharmaceutical compositions of the present invention.

The present invention further encompasses methods for treating or preventing a neurologic dysfunction in a subject, comprising administering to the subject a therapeutically effective amount of the pharmaceutical compositions of the present invention. The present invention also provides methods for post-stroke neuroprotection in a subject by administering a therapeutically effective amount of the pharmaceutical compositions of the present invention.

The present invention further provides kits for use in treating or preventing hyperexcitability disorders in a subject comprising a therapeutically effective amount of the pharmaceutical compositions of the present invention, optionally, in combination with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of the pharmaceutical compositions of the present invention.

Finally, the present invention also provides kits for use in post-stroke neuroprotection in a subject, comprising a therapeutically effective amount of the pharmaceutical compositions of the present invention.

Additional aspects of the present invention will be apparent in view of the detailed description which follows.

### BRIEF DESCRIPTION OF THE FIGURES

FIG. 1 depicts a representation of PKA and β1-subunit regulation of BKCa channel. (A) Signaling through the BKCa-associated β2AR leads to cAMP generation, PKA phosphorylation of S872 (mS1o), and increased channel activity. Increased channel activity may be due to Gα association. B) β1 subunit modification of BKCa channel leads to activation. β1 is denoted as red.

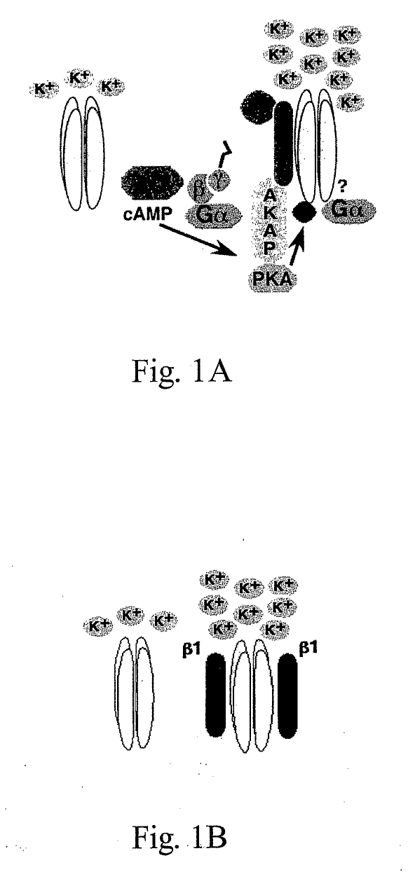

FIG. 2 shows a schematic of the structure of a subunit of BKCa channel. The α subunit is the pore forming subunit; the tetrameric channel is formed by four a subunits. Seven transmembrane domains are shown; S0-S6. The pore is between S5 and S6. The channel has a unique C-terminus, with four additional, non-transmembrane hydrophobic regions (S7-S10). The Ca²⁺ regulatory domains are indicated; the Ca²⁺ bowl, M513 and D362/D367 form independent high affinity Ca²⁺ sensors. The RCK1 and RCK2 domains are indicated. Adapted from (Magleby, K. L., Gating mechanism of BK (Slo1) channels: so near, yet so far. J. Gen. Physiol., 2003. 121(2):81-96).

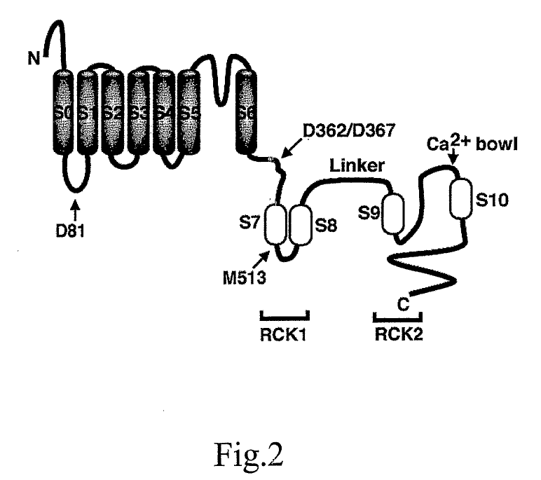

FIG. 3 is a schematic representation of the role of RyR in regulation of SMC constriction and dilation. Local Ca²⁺release (sparks) from RyR activate BKCa, whose outward current (spontaneous transient outward currents; STOC) hyperpolarize the membrane and inhibit voltage gated Ca²⁺ channels (Caᵥ1.2). Agents that increase cAMP in VSMC cause vasodilatation. PKA has a direct effect on BKCa, but also increases spark activity, potentially by increased phosphorylation of the voltage gated Ca²⁺ channel, RyR, and phospholamban (adapted from (Porter, et al., Frequency modulation of Ca²⁺ sparks is involved in regulation of arterial diameter by cyclic nucleotides. Am. J. Physiol., 1998. 274 (Cell Physiol. 43):C1346-C1355)). The presence of the IP3R on the SR is not shown. The β2AR is shown associated with BKCa and Caᵥ1.2, whereas βP1AR does not associate with either channel. The inventors will explore the relationship of PKA phosphorylation of RyR2 and BKCa in Aim 1, utilizing the RyR S2809A genetically altered mice. The regulation of BKCa and RyR by other kinases is not shown.

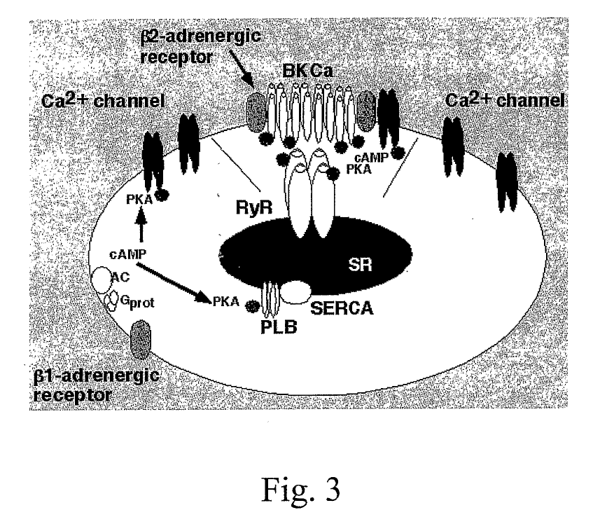

FIG. 4 depicts an electrophysiologic characterization of rottlerin. (A-B) Representative current traces from whole cell patch with 5 mM EGTA in patch pipette. Rottlerin (0.5 μM) was applied to extracellular side through local perfusion. Voltage steps are shown at the right of each tracing; note different maximum voltage steps +200 mV (upper) vs. +120 mV (lower). Rottlerin significantly prolonged tail currents indicative of slowing of deactivation. Tail currents are in opposite direction due to final voltage step (+60 upper, −60 mV left). (C) G-V curves were constructed for indicated conditions utilizing tail analysis (Xia, et al., Multiple regulatory sites in large-conductance calcium-activated potassium channels. Nature, 2002. 418(6900):880-4). Solid lines were fitted with Boltzmann function (Origin 7.5). Exposure to rottlerin shifted G-V curve in the hyperpolarizing direction (indicative of activation). Representative of>30 similar experiments with rat and murine (pictured) s1o. (D-E) Stable mslo HEK293 cells were studied in whole cell configuration with pipette solution containing 5 mM EGTA (˜0 free Ca²⁺). Local perfusion of rottlerin (extracellular; 0.5 M) activated channel (left). Exposure of cell to TEA (5 mM) reversibly inhibited rottlerin induced BKCa activation.

FIG. 5 shows that Intracellular exposure of rottlerin through dialysis for extended period has minimal effect on BKCa channel activity. (A) Representative current traces 1 min and 25 minutes after establishing whole cell voltage patch clamp, dialyzed with 5 mM EGTA (˜0 cytosolic Ca²⁺) and 20 μM rottlerin in patch pipette. Over 25 min, intracellular dialysis of rottlerin had minimal effect on BKCa current. (B-C) After 25 min, the cell was exposed to rottlerin (0.5 μM via local perfusion, which significantly increased channel activity as shown in diary plot (B) and G-V curve (C). Current in diary plot represents maximal current in ramp protocol at +70 mV. Holding potential of ramp was −20 mV, with ramp from −30 mV to +180 mV over 500 ms. G-V curve in (C) generated from tail analysis as described in FIG. 4. Exposure of rottlerin through cytoplasmic administration had minimal effect on channel; only exposure through extracellular space caused activation, suggesting that compound requires access to privileged space only accessible extracellularly.

FIG. 6 shows single channel recordings of BKCa channel. (A) Representative outside-out single charnel traces demonstrating activation of BKCa from local perfusion of rottlerin (0.5 μM) to the extracellular side. Amplitude histograms are shown on the right. Rottlerin does not significantly change the channel conductance. Ca²⁺ was maintained at 0 (virtual; actual<20 nM) through dialysis using patch pipette. Since cellular compartments are excluded from patch and outside-out patch perfused locally with 0 Ca²⁺, activation is Ca²⁺-independent. Channel activation can be inhibited by iberiotoxin or TEA (not shown). (B) Representative inside-out single channel traces demonstrating activation of BKCa with local perfusion of rottlerin (0.5 μM) in presence of 0 Ca²⁺. Amplitude histograms are shown on the right.

FIG. 7 shows that rottlerin activates BKCa channel in HEK and VSMC. (A) Comparison of effects of NS-1619 and rottlerin. Time course of whole cell voltage clamp experiment in stably transfected mSlo HEK293 cell demonstrating current at +60 mV. Current was monitored with ramp protocol; holding potential −60 mV, with ramp from −80 mV to +150 mV over 500 ms. Extracellular application of NS-1619 (10 μM) increased current as previously described (Olesen, et al., Selective activation of Ca(2+)-dependent K+channels by novel benzimidazolone. Eur. J. Pharmacol., 1994. 251(1):53-9). After stabilization of the current, rottlerin (0.5 μM) was applied to the cell by local perfusion. Rottlerin shifted the V_{0.5} by˜100 mV after 5 min. Analysis performed using tail analysis with normalization as previously described (Xia, et al., Multiple regulatory sites in large-conductance calcium-activated potassium channels. Nature, 2002. 418(6900):880-4). (B) HEK cells co-expressing α and β1 subunits were studied in whole cell configuration with 5 mM EGTA (intra-pipette). Rottlerin (0.5 μM) significantly shifted the I-V curve to the left, similar to results in HEK cells expressing only the a subunit (FIG. 5). (C) Human VSMC were studied in outside-out configuration, utilizing single channel recordings, recorded at +60 mV. Exposure of the same patch to rottlerin (0.5 βM) by local perfusion significantly increased Po and open dwell time. Identification of the BKCa channel was determined by conductance and inhibition by iberiotoxin and TEA. (D) Rottlerin inhibits phenylephrine (PE) induced modulation of VSMC tone. Murine femoral arterial rings from were isolated and placed in a wire myograph. PE induced constriction of the vessel was significantly inhibited by rottlerin (0.5 μM). Rottlerin's effects were inhibited by TEA. Figure representative of 4 similar experiments. Error bars are SEM; * p<0.001.

### DETAILED DESCRIPTION OF THE INVENTION

As described above, BKCa channels play a critical role in modulating neuronal processes and smooth muscle contractile tone. Accordingly, BKCa regulation has significant implications in the study of diseases in which smooth muscle contraction may be abnormal. Alteration of the channel's activity by phosphorylation represents an important regulatory pathway leading to modulation of cellular excitability. The inventors of the present invention have herein demonstrated that pharmacologic approaches to activate BKCa channels represent an emerging novel strategy to control membrane excitability.

The present invention relates to several findings concerning BKCa regulation. In particular, the inventors have discovered that rottlerin dramatically increases BKCa channel activity in a non-Ca²⁺dependent, but reversible fashion. Moreover, rottlerin's mechanism appears unique; tail currents are markedly prolonged after exposure to rottlerin, implying a slowing of deactivation and the G-V curve is reversibly shifted by more than 100 mV to the left. Similar results were observed in a rat BKCa channel heterologously expressed in HEK293.

Accordingly, the present invention provides compositions and methods for regulating the BK channel using rottlerin and derivatives thereof. More specifically, the present invention encompasses compositions and methods for treating or preventing BK channel mediated disorders by administering to a subject an effective amount of a BK channel activator, including but not limited to rottlerin and derivatives thereof.

As used herein, a BK channel or BKCa channel mediated disorder refers to disorders related to under or over activation of the BK channel. For purposes of the present invention, such disorders include, but are not limited to, hypertension, asthma, urinary incontinence, gastroenteric hypernotility, coronary spasm, pulmonary disease, psychoses, convulsion, anxiety, erectile dysfunction and neurologic dysfunction.

The term “derivative” as used herein refers to a chemical compound that is structurally similar to another and may be theoretically derivable from it, but differs slightly in composition. For example, an analogue of rottlerin is a compound that differs slightly from rottlerin (e.g., as in the replacement of one atom by an atom of a different element or in the presence of a particular functional group), and may be derivable from rottlerin.

In one aspect of the present invention, a compound for use in modulating BK channel activity is provided having the general formula:

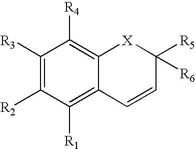

wherein X is CH2, O, N, or S; R1 and R3 is H, OH, NH or SH; R2 is ethanone, acetyl, alkenyl, aryl or alkyl; R4 is CO-[(E)CHCH]n-Ph, CN-[(E)CHCH]n-Ph, or COOZ, wherein Z is alkenyl, aryl, or alkyl; R5 and R6 is H, OH, NH, SH, alkenyl, aryl, or alkyl. In one embodiment, the compound is rottlerin or a derivative thereof.

The present invention also provides a pharmaceutical composition comprising the above-described compound, or a derivative thereof, and optionally, a pharmaceutically acceptable carrier, for use in treating or preventing a BK channel associated disorder. In a specific embodiment, the compound is rottlerin.

The term “treating,” as used herein includes treating any one or more of the conditions underlying or characteristic of a particular disorder. As used herein, the term “preventing” includes preventing the initiation of a particular disorder, delaying the initiation the disorder, preventing the progression or advancement of the disorder, slowing the progression or advancement of the disorder, delaying the progression or advancement of the disorder, and reversing the progression of the disorder from an advanced to a less advanced stage.

By way of example, in an embodiment of the invention, hypertension is treated in a subject in need of treatment by administering to the subject a therapeutically effective amount of rottlerin or a derivative thereof, effective to treat the hypertension. The subject is preferably a mammal (e.g., humans, domestic animals, and commercial animals, including cows, dogs, monkeys, mice, pigs, and rats), and is most preferably a human. The term “therapeutically effective amount” or “effective amount” as used herein refers to the quantity of the compound or pharmaceutical composition according to the invention which is necessary to prevent, cure, ameliorate or at least minimize the clinical impairment, symptoms or complications associated with a particular disorder in either a single or multiple dose. The amounts of the compound or pharmaceutical composition effective to treat or prevent the disorder will vary depending on the particular factors of each case including, but not necessarily limited to, the particular disorder, the stage or severity of the disorder, the subject's weight, the subject's condition and the method of administration. The skilled artisan can readily determine these amounts.

In another aspect of the present invention, the above-described compounds and pharmaceutical compositions can be used to regulate membrane excitability both in vitro and in vivo. In one example, the compounds and pharmaceutical compositions of the present invention can be used to treat or prevent a hyperexcitability disorder. In an embodiment of the invention, the hyperexcitability disorder is asthma. In another embodiment of the invention, the hyperexcitability disorder is hypertension. In other embodiments of the present invention, the hyperexcitability disorder includes, but is not necessarily limited to, urinary incontinence, gastroenteric hypermotility, coronary spasm, psychoses, convulsion and anxiety. In another embodiment, the compounds and pharmaceutical compositions of the present invention are used in treating or preventing erectile dysfunction. In yet other embodiments, the compounds and pharmaceutical inventions of the present invention are used in treating or preventing coronary artery vasospasm. In another embodiment, the compounds and pharmaceutical inventions of the present invention are used in treating or preventing neurologic dysfunction. In another embodiment, the compounds and pharmaceutical inventions of the present invention are used in post-stroke neuroprotection.

The present invention also provides methods for treating or preventing a hyperexcitability disorder in a subject, comprising administering to the subject a therapeutically effective amount of the pharmaceutical composition of the invention. In an embodiment of the invention, the hyperexcitability disorder includes, but is not necessarily limited to, asthma, urinary incontinence, gastroenteric hypermotility, hypertension, coronary spasm, psychoses, convulsion and anxiety.

The present invention also provides methods for treating or preventing erectile dysfunction in a subject by administering to the subject a therapeutically effective amount of the pharmaceutical compositions of the invention. Additionally, the present invention also provides methods for treating or preventing a coronary artery vasospasm in a subject, comprising administering to the subject a therapeutically effective amount of the pharmaceutical compositions of the present invention.

The present invention additionally provides methods for treating or preventing hypertension in a subject, comprising administering to the subject a therapeutically effective amount of the pharmaceutical compositions of the present invention. As used in the context of the present invention, hypertension refers to a condition characterized by an increased systolic and/or diastolic blood pressure. By way of non-limiting example, hypertension in a human subject is characterized by a systolic pressure above 140 mm Hg and/or a diastolic pressure above 90 mm Hg.

The present invention also provides methods for treating or preventing a neurologic dysfunction in a subject, comprising administering to the subject a therapeutically effective amount of the pharmaceutical compositions of the present invention. The present invention also provides methods for post-stroke neuroprotection in a subject by administering a therapeutically effective amount of the pharmaceutical compositions of the present invention.

The present invention further provides kits for use in treating or preventing hyperexcitability disorders comprising an effective amount of the pharmaceutical compositions of the present invention, optionally, in association with a pharmaceutically acceptable carrier. In an embodiment, the hyperexcitability disorder includes, but is not necessarily limited to, asthma, urinary incontinence, gasiroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of the pharmaceutical compositions of the present invention.

Finally, the present invention also provides kits for use in post-stroke neuroprotection in a subject, comprising a therapeutically effective amount of the pharmaceutical compositions of the present invention.

Rottlerin (5,7-dihydroxy-2,2-dimethyl-6-(2,4,6-trihydroxy-3-methyl-5-acetylbenzyl)-8-cinnamoyl-1,2-chromene), and derivatives thereof, have been frequently used as PKCδ inhibitors. The present invention establishes for the first time that rottlerin and its derivatives can also be used to activate the BKCa channel. This new therapy will provide unique strategies to treat and prevent a variety of disorders mediated by BKCa channel activity.

Methods of preparing rottlerin and its derivatives are well known in the art. Rottlerin, for example, is commercially available from A. G. Scientific, Inc., 6450 Lusk Blvd. Suite E102, San Diego, Calif. 92121. Rottlerin and derivatives thereof may be synthesized in accordance with known organic chemistry procedures that are readily understood by those of skill in the art. The term “synthesize” as used in the present invention refers to formation of a particular chemical compound from its constituent parts using synthesis processes known in the art. Such synthesis processes include, for example, the use of light, heat, chemical, enzymatic or other means to form particular chemical composition.

In a method of the present invention, a composition comprising rottlerin or a derivative thereof is administered to a subject in combination with another BKCa channel activator, such that a synergistic therapeutic effect is produced. A “synergistic therapeutic effect” refers to a greater-than-additive therapeutic effect which is produced by a combination of two therapeutic agents, and which exceeds that which would otherwise result from individual administration of either therapeutic agent alone. For instance, administration of rottlerin in combination with a derivative thereof unexpectedly results in a synergistic therapeutic effect by providing greater efficacy than would result from use of either of therapeutic agents alone. Rottlerin enhances the effect of the rottlerin derivative. Therefore, lower doses of one or both of therapeutic agents may be used in treating for example, hypertension, resulting in increased therapeutic efficacy and decreased side-effects.

In the method of the present invention, administration of a composition comprising rottlerin or a derivative thereof “in combination with” another BKCa channel activator refers to co-administration of the two therapeutic agents. Co-administration may occur concurrently, sequentially, or alternately. Concurrent co-administration refers to, for example, administration of both the rottlerin composition and the rottlerin derivative at essentially the same time. For concurrent co-administration, the courses of treatment with rottlerin or a derivative thereof and with another BKCa channel activator may be run simultaneously. For example, a single, combined formulation, containing both an amount of rottlerin and an amount of a rottlerin derivative in physical association with one another, may be administered to the subject. The single, combined formulation may consist of an oral formulation, containing amounts of both the rottlerin and the rottlerin derivative, which may be orally administered to the subject, or a liquid mixture, containing amounts of both the rottlerin and the rottlerin derivative, which may be injected into the subject.

It is also within the confines of the present invention that an amount of one rottlerin derivative and an amount of a general rottlerin derivative may be administered concurrently to a subject, in separate, individual formulations. Accordingly, the method of the present invention is not limited to concurrent co-administration of the BKCa channel activators in physical association with one another.

In the method of the present invention, the rottlerin and the rottlerin derivative also maybe co-administered to a subject in separate, individual formulations that are spaced out over a period of time, so as to obtain the maximum efficacy of the combination. Administration of each therapeutic agent may range in duration from a brief, rapid administration to a continuous perfusion. When spaced out over a period of time, co-administration of the rottlerin and the rottlerin derivative may be sequential or alternate. For sequential co-administration, one of therapeutic agents is separately administered, followed by the other. For example, a full course of treatment with the rottlerin composition may be completed, and then may be followed by a full course of treatment with the rottlerin derivative composition. Alternatively, for sequential co-administration, a full course of treatment with the rottlerin composition may be completed, then followed by a full course of treatment with the rottlerin derivative composition. For alternate co-administration, partial courses of treatment with the rottlerin derivative composition may be alternated with partial courses of treatment with the rottlerin composition, until a full treatment of each therapeutic agent has been administered.

Therapeutic agents of the present invention (either in separate, individual formulations or in a single combined formulation) may be administered to a human or animal subject by known procedures including, but not limited to, oral administration, parenteral administration (e.g., intramuscular, intraperitoneal, intravascular, intravenous, or subcutaneous administration), and transdermal administration. Preferably, therapeutic agents of the present invention are administered orally or intravenously.

For oral administration, the formulations of the BKCa channel activators either alone or in combination may be presented as capsules, tablets, powders, granules, or as a suspension. Tihe formulations may have conventional additives, such as lactose, mannitol, corn starch, or potato starch. The formulations also may be presented with binders, such as crystalline cellulose, cellulose derivatives, acacia, corn starch, or gelatins. Additionally, the formulations may be presented with disintegrators, such as corn starch, potato starch, or sodium carboxymethyl cellulose. The formulations also may be presented with dibasic calcium phosphate anhydrous or sodium starch glycolate. Finally, the formulations may be presented with lubricants, such as talc or magnesium stearate.

For parenteral administration, the formulations of the BKCa channel activator either alone or in combination with another different BKCa channel activator may be combined with a sterile aqueous solution which is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. Moreover, the formulations may be delivered by any mode of injection, including, without limitation, epifascial, intracapsular, intracutaneous, intramuscular, intraorbital, intraperitoneal (particularly in the case of localized regional therapies), intraspinal, intrasternal, intravascular, intravenous, parenchymatous, or subcutaneous.

For transdermal administration, the formulations of the BKCa channel activator (whether individual or combined) may be combined with skin penetration enhancers, such as propylene glycol, polyethylene glycol, isopropanol, ethanol, oleic acid, N-methylpyrrolidone, and the like, which increase the permeability of the skin to therapeutic agent, and permit therapeutic agent to penetrate through the skin and into the bloodstream. Therapeutic agent/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent such as methylene chloride, evaporated to the desired viscosity, and then applied to backing material to provide a patch.

The dose of the BKCa channel activator of the present invention may also be released or delivered from an osmotic mini-pump. The release rate from an elementary osmotic mini-pump may be modulated with a microporous, fast-response gel disposed in the release orifice. An osmotic mini-pump would be useful for controlling release, or targeting delivery, of therapeutic agents.

It is within the confines of the present invention that the formulations of the BKCa channel activator may be further associated with a pharnaceutically-acceptable carrier, thereby comprising a pharmaceutical composition. The pharmaceutically-acceptable carrier must be “acceptable” in the sense of being compatible with the other ingredients of the composition, and not deleterious to the recipient thereof. Examples of acceptable pharmaceutical carriers include, but are not limited to, carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage.

The formulations of the present invention may be prepared by methods well-known in the pharmaceutical art. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering therapeutic agents of the present invention (i.e., rottlerin and derivatives thereof, either in separate, individual formulations, or in a single, combined formulation) to a subject to treat a BKCa channel mediated disorder. Therapeutic agents are provided in amounts that are effective to treat or prevent heart failure in the subject. These amounts may be readily determined by the skilled artisan.

In the synergistic combination of the present invention, the BKCa channel activator may be combined in a single formulation, such that, for example, the amount of the rottlerin composition is in physical association with the amount of the rottlerin derivative composition. This single, combined formulation may consist of an oral formulation, containing amounts of both the BKCa channel activators, which may be orally administered to the subject, or a liquid mixture, containing amounts of both the BkCa channel activators, which may be injected into the subject.

Alternatively, in the synergistic combination of the present invention, a separate, individual formulation of a BKCa channel activator may be combined with a separate, individual formulation of a different BKCa channel activator. For example, an amount of a rottlerin derivative may be packaged in a vial or unit dose, and an amount of the rottlerin derivative may be packaged in a separate vial or unit dose. A synergistic combination of the rottlerin and the rottlerin derivative then may be produced by mixing the contents of the separate vials or unit doses in vitro. Additionally, a synergistic combination of the rottlerin and rottlerin derivative may be produced in vivo by co-administering to a subject the contents of the separate vials or unit doses, according to the methods described above. Accordingly, the synergistic combination of the present invention is not limited to a combination in which amounts of the BKCa channel activators are in physical association with one another in a single formulation.

For example, the BKCa channel activator rottlerin can be administered in a dosage of about 0.5 mg/kg/day to 25 mg/kg/day. Preferably, rottlerin is administered in a dosage of about 5 mg/kg/day to about 10 mg/kg/day. Similarly, effective rottlerin derivatives can be administered in a dosage of about 0.5 mg/kg/day to 25 mg/kg/day. Preferably, an effective rottlerin derivative is administered in a dosage of about 5 mg/kg/day to about 10 mg/kg/day. The appropriate effective therapeutic amounts of the BKCa channel activators within the listed ranges can be readily determined by the skilled artisan.

The invention also provides compositions and methods for treating or preventing neuronal damage in a post-stroke subject comprising administering to the subject a therapeutically effective amount of rottlerin or derivatives thereof.

The present invention further provides kits for use in treating or preventing hyperexcitability disorders in a subject comprising a therapeutically effective amount of the pharmaceutical compositions of the present invention, optionally, in combination with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of the pharmaceutical compositions of the present invention. The present invention further provides kits for use in treating or preventing hyperexcitability disorders in a subject comprising a therapeutically effective amount of the pharmaceutical compositions of the present invention, optionally, in combination with a pharmaceutically acceptable carrier. In an embodiment, the hyperexcitability disorder includes, but is not necessarily limited to, asthma, urinary incontinence, gastroenteric hypermotility, hypertension, coronary spasm, psychoses, convulsion and anxiety.

The present invention also provides kits for use in treating or preventing erectile dysfunction, coronary artery vasospasm, hypertension or neurologic dysfunction in a subject, comprising administering a therapeutically effective amount of the pharmaceutical compositions of the present invention.

Finally, the present invention also provides kits for use in post-stroke neuroprotection in a subject, comprising a therapeutically effective amount of the pharmaceutical compositions of the present invention.

The present invention is described in the following Examples, which are set forth to aid in the understanding of the invention, and should not be construed to limit in any way the scope of the invention as defined in the claims which follow thereafter.

### EXAMPLES

### Example 1

While the foregoing invention has been described in some detail for purposes of clarity and understanding, it will be appreciated by one skilled in the art, from a reading of the disclosure, that various changes in form and detail can be made without departing from the true scope of the invention in the appended claims.

### Rottlerin Activates BKCa Channel

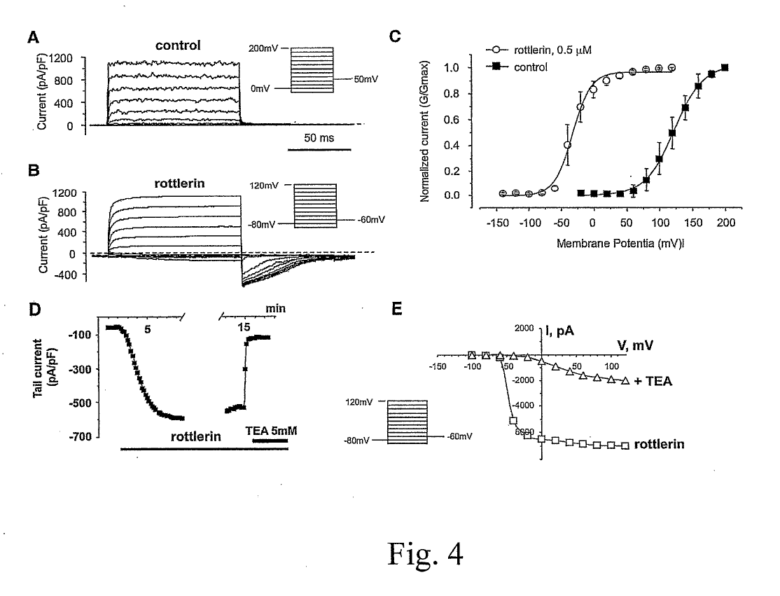

BKCa regulation has significant implications in the study of diseases in which smooth muscle contraction may be abnormal. BKCa can be potently regulated by PKC activating vasoconstrictors. In order to elucidate the functional effects of PKC phosphorylation, the inventors evaluated putative PKC inhibitory compounds under non-phosphorylated conditions (should have no effect). Surprisingly, one compound, rottlerin (≦100 nM) dramatically increased channel activity (FIG. 4) in a non-Ca²+ dependent, but reversible fashion. No other PKC inhibitor had any effect on BKCa channel activity under basal conditions (not shown). Moreover, rottlerin's mechanism appears unique; tail currents are markedly prolonged after exposure to rottlerin, implying a slowing of deactivation and the G-V curve is reversibly shifted by more than 100 mV to the left (FIG. 4B). Similar results were observed in a rat BKCa channel heterologously expressed in HEK293.

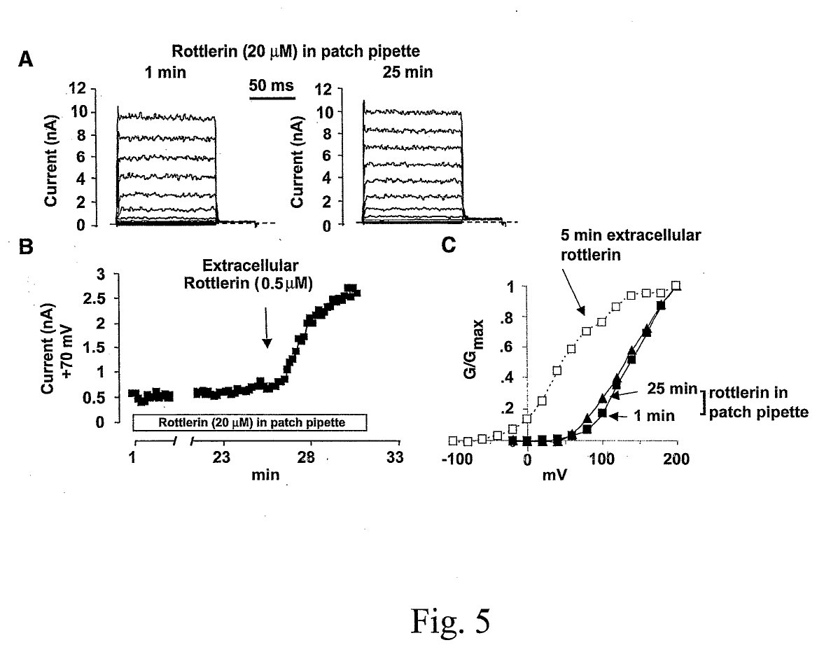

Intracellular dialysis of rottlerin (the inventors tested up to 20 μM, which represents˜200 fold more than the maximum extracellular concentration tested) had only a relatively small effect on the channel activity, suggesting that access to the activating site requires extracellular exposure (FIG. 5).

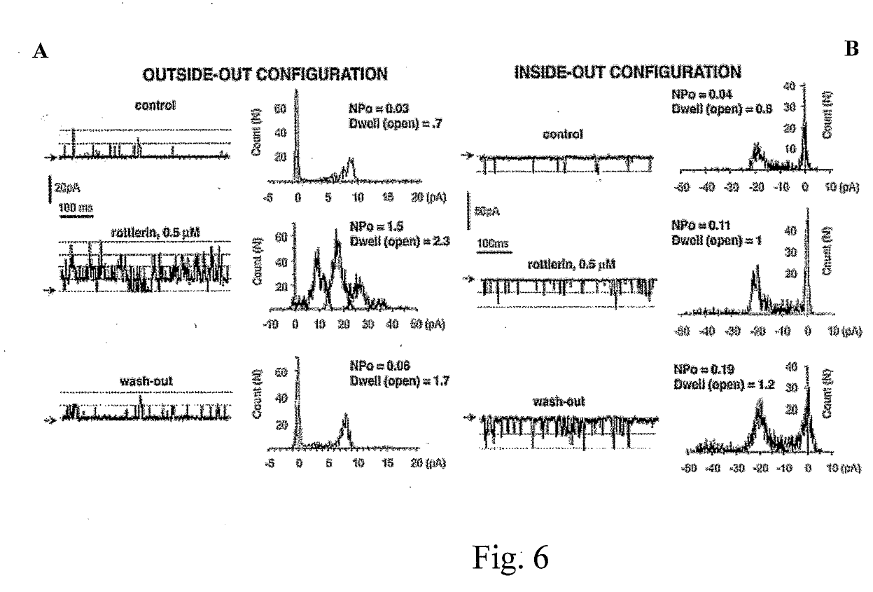

Although rottlerin has been proposed to have other effects at significantly high concentrations (˜10 μM), the BKCa activation is not due to modulation by PKC (or other cellular components), as it can be demonstrated using a cell-free configuration (FIG. 6).

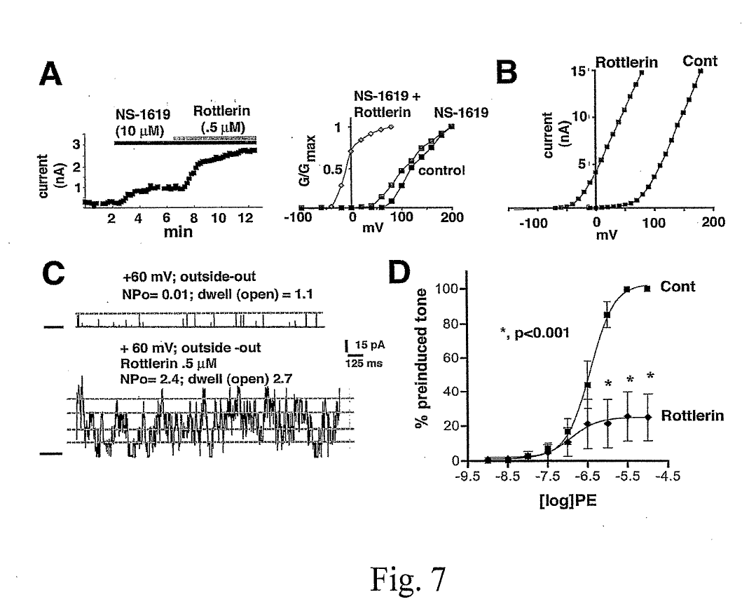

Rottlerin was compared to one of the originally described BKCa channel activators, NS-1619 (Olesen, et al., Selective activation of Ca(2+)-dependent K+ channels by novel benzimidazolone. Eur. J. Pharmacol., 1994. 251(1):53-9). NS-1619 (10 μM) activated peak K⁺ current in HEK293 cells stably transfected with mSlo (FIG. 7A); addition of rottlerin (0.5 μM) after NS-1619 administration incrementally increased current in 0 Ca²⁺. Co-expression of the β1 subunit does not modify the effects of rottlerin; in HEK293 cells expressing both mSlo and β1 subunit, rottlerin activated the channel (FIG. 17B). Given rottlerin's potent BKCa activating effects, in the absence of Ca²⁺and the presence of the β1 subunit, the inventors hypothesized that rottlerin may be effective in mediating the relaxation of vascular smooth muscle. Human vascular smooth muscle cells (VSMC) grown in vitro express BKCa channels. Single channel recordings (outside-out configuration) demonstrate that rottlerin activated BKCa channels (FIG. 7C) by increasing Po and open dwell time. Next, the inventors determined whether rottlerin could mediate vascular relaxation, as demonstrated for several other BKCa channel activators (Nardi, et al., Natural modulators of large-conductance calcium-activated potassium channels. Planta. Med., 2003. 69(10):885-92). Rottlerin (4 μM) reduced phenylephrine mediated contraction by>50% (FIG. 7D), although 1 μM had no significant effect. The rottlerin mediated effect was inhibited by TEA, suggesting a prominent K⁺ channel contribution to the blunting of contractile tone. The difference in concentration between the BKCa channel effects in electrophysiologic experiments compared to vascular rings may be explained by the hydrophobic properties of the compound and the experimental conditions. Interestingly, NS-1619's efficacy in mediating vascular relaxation was diminished in a hypertensive rat model (Callera, et al., Ca2+-activated K+ channels underlying the impaired acetylcholine-induced vasodilation in 2K-1C hypertensive rats. J. Pharmacol. Exp. Ther., 2004. 309(3):1036-42), compared to controls, perhaps consistent with the down-regulation of the β1, but not α subunit observed in hypertensive animal models (Amberg, et al., Downregulation of the BK channel betal subunit in genetic hypertension. Circ. Res., 2003.93(10)!965-71; Amberg, et al., Modulation of the molecular composition of large conductance, Ca(2+) activated K(+) channels in vascular smooth muscle during hypertension. J. Clin. Invest., 2003. 112(5):717-24). Based upon rottlerin's efficacy in in vitro electrophysiologic experiments (FIGS. 4-7), in low (Ca²⁺)ᵢ and in the absence of a β1 subunit, the inventors hypothesize that rottlerin-like compounds may be more effective.

What is claimed is:

1. A compound for use in modulating BK channel activity having the general formula: 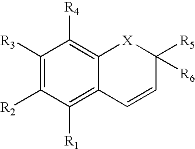
wherein X is selected from the group consisting of CH2, O, N, and S; R1 and R3 are selected from the group consisting of H, OH, NH and SH; R2 is selected from the group consisting of ethanone, acetyl, alkenyl, aryl and alkyl; R4 is CO-[(E)CHCH]n-Ph, CN-[(E)CHCH]n-Ph, or COOZ, wherein Z is selected from the group consisting of alkenyl, aryl, and alkyl; R5 and R6 are selected from the group consisting of H, OH, NH, SH, alkenyl, aryl, and alkyl.

2. The compound of claim 1, wherein the compound is rottlerin.

3. A pharmaceutical composition comprising the compound of claim 1, or a derivative thereof, and optionally, a pharmaceutically acceptable carrier, for use in treating or preventing a BK channel mediated disorder.

4. The pharmaceutical composition of claim 3, wherein the compound is rottlerin.

5. The compound of claim 3 or 4 for use in regulating membrane excitability.

6. The pharmaceutical composition of claim 3 or 4 for use in treating or preventing a hyperexcitablility disorder.

7. The method of claim 3 or 4, wherein the hyperexcitability disorder is selected from the group consisting of asthma, urinary incontinence, gastroenteric hypermotility, hypertension, coronary spasm, psychoses, convulsion and anxiety.

8. The pharmaceutical composition of claim 3 or 4, for use in treating or preventing erectile dysfunction.

9. The pharmaceutical composition of claim 3 or 4, for use in treating or preventing coronary artery vasospasm.

10. The pharmaceutical composition of claim 3 or 4, for use in treating or preventing hypertension.

11. The pharmaceutical composition of claim 3 or 4, for use in treating or preventing neurologic dysfunction.

12. The pharmaceutical composition of claim 3 or 4, for use in post-stroke neuroprotection.

13. A method for treating or preventing a hyperexcitability disorder in a subject, comprising administering to the subject a therapeutically effective amount of the pharmaceutical composition of claim 3 or 4.

14. The method of claim 11, wherein the hyperexcitability disorder is selected from the group consisting of: asthma, urinary incontinence, gastroenteric hypermotility, hypertension, coronary spasm, psychoses, convulsion and anxiety.

15. A method for treating or preventing a erectile dysfunction in a subject, comprising administering to the subject a therapeutically effective amount of the pharmaceutical composition of claim 3 or 4.

16. A method for treating or preventing a coronary artery vasospasm in a subject, comprising administering to the subject a therapeutically effective amount of the pharmaceutical composition of claim 3 or 4.

17. A method for treating or preventing a hypertension in a subject, comprising administering to the subject a therapeutically effective amount of the pharmaceutical composition of claim 3 or 4.

18. A method for treating or preventing a neurologic dysfunction in a subject, comprising administering to the subject a therapeutically effective amount of the pharmaceutical composition of claim 3 or 4.

19. A method for post-stroke neuroprotection in a subject, comprising administering to the subject a therapeutically effective amount of the pharmaceutical composition of claim 3 or 4.

20. A kit for use in treating or preventing a hyperexcitability disorder in a subject, comprising a therapeutically effective amount of the pharmaceutical composition of claim 3 or 4.

21. The method of claim 20, wherein the hyperexcitability disorder is selected from the group consisting of: asthma, urinary incontinence, gastroenteric hypermotility, hypertension, coronary spasm, psychoses, convulsion and anxiety.

22. A kit for use in treating or preventing a erectile dysfunction in a subject, comprising a therapeutically effective amount of the pharmaceutical composition of claim 3 or 4.

23. A kit for use in treating or preventing a coronary artery vasospasm in a subject, comprising a therapeutically effective amount of the pharmaceutical composition of claim 3 or 4.

24. A kit for use in treating or preventing a hypertension in a subject, comprising a therapeutically effective amount of the pharmaceutical composition of claim 3 or 4.

25. A kit for use in treating or preventing a neurologic dysfunction in a subject, comprising a therapeutically effective amount of the pharmaceutical composition of claim 3 or 4.

26. A kit for use in post-stroke neuroprotection in a subject, comprising a therapeutically effective amount of the pharmaceutical composition of claim 3 or 4.
